(19) United States
(12) Patent Application Publication
(10) Pub. No.: US 2006/0008511 A1
(43) Pub. Date: Jan. 12, 2006

(54) Probiotic products for pet applications

(76) Inventors: Jhy-Jhu Lin, Potomac, MD (US); Jolinta Y. Lin, Potomac, MD (US)

Correspondence Address: William S. Ramsey, 5253 Even Star Place, Columbia, MD, 21044, US

(21) Appl. No.: 11/177,264

(22) Filed: Jul. 7, 2005

Related U.S. Application Data
(60) Provisional application No. 60/585,941, filed on Jul. 8, 2004.

(51) Int. Cl. A61K 45/00; C12N 1/20

(52) U.S. Cl. 424/442; 424/93.45; 435/252.9

(57) ABSTRACT

An exemplary embodiment providing one or more improvements includes feeding pets with probiotic microbes encapsulated in a mixture of xanthan gum and chitosan, or in gelatin, specifically Pediococcus acidilactici and Saccharomyces boulardii. Such encapsulation protects the viability of the probiotic microbes against unfavorable temperatures. Such feeding has the benefit of reducing odors, and improving digestion in pets which have these problems.

### CROSS-REFERENCE(S)

This application claims priority from provisional application Ser. No. 60/585,941 filed Jul. 8, 2004.

### STATEMENT REGARDING FEDERALLY SPONSORED RESEARCH OR DEVELOPMENT

Not Applicable.

### REFERENCE TO A “MICROFICHE appendix.”

Not Applicable.

### BACKGROUND

Description of Related Art Including Information Disclosed Under 37 CFR 1.97 and 37 CFR 1.98.

U.S. Pat. No. 5,968,569 discloses a pet food product of a gelatinized starch matrix including a probiotic micro-organism. Specifically disclosed are Saccharomyces and Pediococcus acidilactici.

U.S. Pat. No. 6,551,633 discloses a milk based powder for pets which includes lactase and lactose. Also disclosed are the probiotic organisms of U.S. Pat. No. 5,968,569.

U.S. Pat. No. 6,780,447 discloses animal foods comprising sorbic acid and live or dead microorganisms. A very large number of species is disclosed including P. acidilactici.

U.S. Pat. No. 6,827,957 discloses animal foods of specific formulation having a soft inner component and a hard shell along with probiotics. Specifically, Saccharomyces is disclosed.

U.S. Pat. No. 6,835,397 discloses an encapsulated yeast including a variety of probiotics including Saccharomyces. boulardii and Pediococcus. acidilactic (sic).

U.S. Pub. Pat. Applic. 2003/0049240 discloses a method for treating helicobacter infections including the use of Lactobacillus and Bifidobacterium.

U.S. Pub. Pat. Applic. 2004/0197352 discloses a prebiotic composition which reduces creatine and BUN and includes a variety of microbial species.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

Embodiments disclosed include a preparation for pets comprising probiotic microbes encapsulated in a mixture of xanthan and chitosan gums. In embodiments the probiotic microbes comprise Saccharomyces yeast and lactic acid bacteria. In embodiments the probiotic microbes comprise yeast. In embodiments the probiotic microbes comprise lactic acid bacteria. In embodiments the yeast is Saccharomyces. In embodiments the lactic acid bacteria is Pediococcus. In embodiments the Saccharomyces yeast is Saccharomyces cereviase boulardii also termed Saccharomyces boulardii. In embodiments the lactic acid bacteria is Pediococcus acidilactici. In embodiments the xanthan gum concentration is from about 0.2 percent weight by volume to about 2 percent weight by volume and the concentration of chitosan gum is about 0.1 percent weight by volume to 1.0 percent weight by volume and the pH is from about 2 to about 7. In embodiments the xanthan gum concentration is from about 01.25 percent weight by volume and the concentration of chitosan gum is about 0.4 percent weight by volume and the pH is about 4.15.

Other embodiments disclosed include the processes of reduction of diarrhea, vomiting, body odor or flatulence in pets in need of such reductions comprising the step of feeding the pet encapsulated probiotic microbes. Other embodiments disclosed include the processes of improvement in appetite, reduced diarrhea, increased firmness of stool, improvement in digestion, improvement of swallowing in pets in need of such reductions comprising the step of feeding the pet gelatin encapsulated probiotic microbes. In other embodiments the probiotic microbes are Pediococcus acidilactici and Saccharomyces cerevisiae boulardii. In other embodiments the probiotic microbes are encapsulated in a mixture of xanthan gum and chitosan and in others the pet is a dog.

The following embodiments and aspects thereof are described and illustrated in conjunction with systems, tool and methods which are meant to be exemplary and illustrative, not limiting in scope. In various embodiments, one or more of the above-described problems have been reduced or eliminated, while other embodiments are directed to other improvements.

In addition to the exemplary aspects and embodiments described above, further aspects and embodiments will become apparent by reference to the drawings and by study of the following descriptions.

### BRIEF DESCRIPTION OF THE DRAWINGS

FIG. 1 shows the relationship between pH and capsule hardness.

FIG. 2 shows the relationship between viability of encapsulated and unencapsulated probiotic microbes and reduced temperature.

FIG. 3 shows the relationship between viability of encapsulated and unencapsulated probiotic microbes and elevated temperature.

FIG. 4 shows the relationship between viability of encapsulated and unencapsulated probiotic microbes and time of exposure to pH 2.

### DETAILED DESCRIPTION

Probiotics are the beneficial living bacteria that naturally exist in the gastrointestinal (GI) tracts of humans and animals. Probiotics are well accepted as the food supplements for human consumptions. When patients have discomforts of digestive systems because of treatment with antibiotics or suffering form travel, doctors often recommend the patients to take probiotics to restore the microflora in patient digestive systems. Recently, the medical community increasingly recognizes probiotics as the agents that are able to enhance human immune responses for improving the efficacy of vaccine and for disease prevention. Probiotics are quickly regarded as one of the primary categories by the functional food industry. In farm animals such as pigs, cattle, dairy cows and poultry, probiotics are widely used as the substitutes for antibiotics as the growth promoters. Producers have recognized the beneficial effects of probiotics that not only improve the animal growth but also reduce the infection of enteric pathogens significantly. The beneficial effects of probiotics on pets (dogs, cats, and other small animals like guinea pigs) have attracted many researchers to investigate the mechanisms, and the research results were published in many journals. Today, pet food manufactures include probiotics as one of the important ingredients in many premium pet foods. Probiotics in capsules or chewable tablets for pet's application are also commercially available. However, pet owners either are not familiar with probiotics or have experiences with the variable Probiotics effects on pets, and have the doubts about the real functions of probiotics. Although the trends for human and farm animals are accepted probiotics as the nutrient supplements or as the powerful neutraceutical products, pet owners are not fully aware that probiotics can contribute significant effects on pets in good health to expand their life span.

How do probiotics function as the beneficial effects on pets? Probiotics have to be able to travel along pet's GI tracts. When they have the opportunity to attach to GI tract surfaces, probiotic microorganisms can start to replicate. When probiotic microorganisms replicate and grow, they will decompose the food token by pets to produce acid compounds, which will create unfavorable acidic environments for most of GI tract pathogens to survive. Some of the probiotics also secrete the toxic compounds that are harmful to the pathogens. Moreover, as the Probiotics attached to pet's GI tracts, they become generic immunogens, raise the antibody production and enhance the pet immune response for pathogen infections. As probiotic microorganisms multiply, they occupy the surfaces of GI tracts and prevent the possibility for the pathogens to attach to pet's GI tracts for infection. During the process of multiplication, Probiotics degrade the complex food compounds into the simple nutrition for pets to absorb and to utilize. This will not only help the pets to strengthen their bodies but also reduce the bad odors typically generated by pets caused either by the incomplete food digestions or by excess gas production through different digestion pathways. Therefore, in order to have the effects of probiotics on pets, the pet owners have to make sure to deliver the live probiotics into pet's GI tracts for microorganisms to multiply and to grow. It is critical to have the sources of viable probiotics for pet to uptake and to ensure the live probiotics that will be able to reach pet's GI tracts in order to make sure that the pet will have the beneficial effects of probiotics.

Let us take a close look of these two critical issues when we apply probiotics to the pets. By understanding these critical issues, we can easily find out why the pet owners experienced the variable effects of probiotics. If we go to pet store, we may easily find many pet foods do include the probiotics, especially, probiotic fermentation cultures. Interestingly, Canadian scientists used to perform the extensive research survey for 19 commercially available pet foods, which claim to contain probiotics. They reported that no products contained all the listed Probiotics, and average bacterial growth only ranged from 0 to 1.8×10⁵ CFU/g (Colony Forming Unit over weight, gm. This is the typical measurable unit for microbiologists to present the amounts of living bacteria in defined weights). The publication is available in Can. Vet. J., 2003, 44:212-215. Furthermore, once pets eat the pet foods, pets secreted many different enzymes to help to digest the foods, which are able to destroy the Probiotics viability too. As the foods move down to pet's GI tracts, probiotics have to go through very acidic and high salts environments, especially, in pet's stomach that can be as low as pH 1.0. Most of Probiotics will not be able to survive through these harsh environments. In fact the survival percentages of live probiotics is so low that one has to do high numbers of live probiotics for daily oral administration to guarantee the beneficial effects. It is well recognized that the daily oral administration of live probiotics has to be greater than 1×10¹⁰ in human or 1×10⁹ CFU in animals to found the beneficial effects of probiotics. If we convert this amount of Probiotics in the best available pet foods described by Canadian scientists, the pets at least have to take more than 10 kg pet foods per day to be able to see the probiotics beneficial effects. Combination of far less numbers of probiotics to feed the pets with the pet natural defense systems in GI tracts, we can easily recognize why the variable effects of probiotics are observed by pet owners. Once pet owners realize to feed the pet with right numbers of live probiotics to the pet, the health benefits of probiotics on the pet will be recognized without doubts.

However, since probiotics are biological entities, delivery of sufficient doses is constantly challenged by inherent factors that might limit their biological activity, including the conditions of growth, processing, preservation, and storage. Specifically, loss of probiotic viability occurs at many distinct stages, including freeze-drying of cells during initial manufacturing, feed preparation (high temperature and high pressure), transportation and storage (temperature fluctuations), and after consumption or in gastrointestinal (GI) track (low pH and bile salts). One of the determined factors for probiotics to have beneficial effects is to maintain the high concentration of viable cells for animals and humans to uptake. Although many commercial probiotic products are available as the additive of animal feed and/or as human functional foods, most of them lost the viability during the manufacture process, transport, storage and animal feed process (Cinto-Cruce and Gould, 2001). Recently, microencapsulation of probiotics using lipids as the carriers has demonstrated the success for improving the probiotics viability (Pacifico et al., 2001). However, there is relatively little information and progress on microencapsulation of probiotics, especially using biopolymers as the microcarriers.

Microencapsulation, extensively used by pharmaceutical, chemical, and food industries to protect precious and/or active ingredients and ensure proper delivery, is limited to the techniques used (emulsion and extrusion) and the composition of microcarriers, including Na-alginate (also in combination with starch, pectin or whey proteins), gum arabic (also known as gum acacia), and K-carrageenan (also in combination with locust bean gum). Not only each of the systems has its own limitations, these common systems usually suffer from low mechanical stability. For instance, although alginate is the most commonly used polymer due to its simplicity, low cost, and excellent biocompatibility, the low mechanical strength of the gel makes it highly susceptible to decalcifying and acidification. The microencapsulation using biopolymers greatly enhance the benefit of probiotics as healthful ingredients by retaining sufficient viability and bioactivity under harsh processing conditions during animal feed and pet food production. In addition to improving the shelf life stability, the transportation costs of these microorganisms will also be reduced if the resulting microcapsules could be stored under room temperature.

Microbial exopolysaccharides are classified as biopolymers and are widely used in foods, medicines, and industrial products (Marin, 1998). Microbial biopolymers, unlike other carriers, are capable of forming a three-dimensional structure that is stabilized by cross-links connecting junction zones between individual molecules (Lo et al., 2003). In nature, for example, Xanthomonas campestris, a plant pathogen of cabbage, produces xanthan gum as an extracellular slimy material to help the cells attach to their host and to endure environmental stresses. Therefore, application of microencapsulation to bacteria using microbial biopolymers provide the new approach to improve the bacterial viability under harsh environmental conditions.

Studies of GI tract infections have shown that probiotics can modulate the immune response to antigens expressed by GI pathogens (Isolauri 2003). When mice were fed L. acidophilus and/or L. casei prior to oral challenge with Salmonella typhimurium, researchers documented that ˜100% of the probiotic-treated group mice survived S. typhimurium challenge compared to <20% survival in control animals. Anti-Salmonella antibody titers were higher in both the serum and GI tract mucosa of the mice fed L. acidophilus/L. casei (Perdigon et al., 1990). Similarly, oral administration of Bifidobacterium breve stimulated an improved IgA response to cholera toxin in mice (Yasui et al., 1992), and L. rhamnosus GG was shown to increase IgA rotavirus-specific antibody secreting cells in children with acute rotavirus diarrhea (Kaila et al., 1992). Both cellular and humoral immune responses were demonstrated when rotavirus-infected piglets were fed B. lactis HN019 (Shu et al., 2001)

Enhanced antibody responses to ovalbumin were demonstrated in gnotobitic mice fed B. bifidum (Moreaue at al., 1990). This indicates that probiotics could be used to stimulate an antigen-specific mucosal immune response, and to provide increased protection to non-mucosal sites. Significant increases in IgG anti-influenza antibodies were observed when B. breve was fed to mice prior oral challenge with influenza vaccine (Yasui et al, 1999). Increased serum IgA titers to Pseudomonas aeruginosa were detected in mice fed with L. casei (Alvaez et al., 2001). IgA, IgG and IgM antibodies against Ecoli and rotavirus were found in the feces of piglets fed Bifidobacterium lactis HN019 (Shu et al., 2001). Recently, local cell-mediated immunity by Lactobacillus-feed, E. acervulina infected broiler chickens was demonstrated based on the higher IL-2 secretion and lower E. acervulina oocyst production by Dalloul et al., 2003. However, few or no reports related to immune responses were described for lactic acid bacteria other than Lactobacillus or Biofidobacterium.

Selection through the survival of feces from probiotics-feed chickens

Strains of lactic acid bacteria differentially stimulate the host immune system. The colonization of the GI tract with probiotic microorganism represents the first step towards establishing a beneficial effect using the introduced bacteria. In order for bacteria to colonize effectively the host, P. acidilactici, it must grow in low pH and bile that represent in the GI tract. The strain selection will be emphasized on the isolation of survival strains from feces collected from P. acidilactici-feed chickens without inoculation of Eimieria. At days 7, 11, 14, 18, and 21, the droppings from P. acidilactici-feed chickens will be collected from three individual chickens. Following the similar procedures performed on the droppings from oocysts production, the droppings will be resuspended and soaked in PBS buffer instead of water. The conventional, microbiological culture for determination of the quantitative numbers of colony formation units (CFU) will be used to isolate the single isolated bacterial colonies and to correlate with the colonization of P. acidilactici in chickens. For colonization evaluation, a series of dilutions of homogenized droppings will be plated onto different selective media (such as: MRS media for P. acidilactici, Rogosa media for Lactobacillus spp, RCA medium for Clostridium spp. LB media for E. coli.) and incubated in different growth conditions. After completing the collection of CFU, hundreds of single colonies isolated from MRS media will be transferred onto new fresh MRS media containing 0.9% bile at pH 2.0, which is regarded as the standard GI tract in humans and animals, for further selection of P. acidilactici. The transfers will be repeated for two more times onto new fresh MRS media containing 0.9% bile at pH 2.0, and the survivals of single colony will be further evaluated by pulse-field gel electrophoresis and API biochemical assays for bacterial strains confirmation before bacteria will be made as the glycerol stock and stored at −70° C.

Strains selection through the colonization of cell lines in vitro

Adhesion of bacteria to the human cell lines Caco-2 and HT29 has been shown to correlate with lactic acid bacterial colonization in animals (Brassart et al., 1998; Tuomola and Salminen 1998). Further selection of strains that are able to grow at 0.9% bile at pH 2.0 will be selected by the co-cultivation of bacteria with the Caco-2 and HT29 cell lines. Determination for bacterial adhesion to Caco-2 and HT29 cell lines will be confirmed by microscopic examination and will be repeated two more times. Bacteria that can grow at 0.9% bile at pH 2.0 and show the adhesion to Caco-2 and HT29 cell lines will be prepared as highly concentrated probiotics at 10 billion/g for chicken feeding in order to do further screening for bacteria with enhanced immune responses in chickens.

Strains selection through oral administration of bacteria to E. maxima vaccinated chickens

To select P. acidilactici strains capable of enhancing the immune response of the colonized host, Bacteria that adhere effectively to the cell lines will be re-selected in bacteria-feed and E. maxima vaccinated chickens. These in vitro and in vivo selection methods should yield P. acidilactici strains with enhanced colonization and immune promoting properties in animal. The chickens will be fed with the selected P. acidilactici strain, vaccinated with E. maxima live oocysts, and infected with high amounts of E. maxima sporulated oocysts. The sample collections and the assays for determination of immune responses and disease infection will be the same. The selection cycle will be repeated one more time to confirm the selected strains that have the enhanced immune response properties.

### EXAMPLE 1

An eight year old black Labrador hybrid with Beagle and Dalmatian, was fed and observed as below. Symptoms: Throw outs or vomiting daily, bad body odors, constantly producing and releasing gas with bad odors or flatulence.

Feeding procedure: daily fed a piece of cheese wrapped with a capsule of MITOMAX, which contains 4 billions CFU of Pediococcus acidilactici and Saccharomyces boulardii, starting from Jun. 21 to Jul. 4, 2004. MITOMAX is a trademark of Imagilin Technology, LLC, Potomac, Md. for gelatin encapsulated probiotics.

TABLE 1

| | | | **Body | ***Bad odors of |
|---|---|---|---|---|
| Date | MITOMAX | *Throws out | odors | gas release |
| 6/19/2004 | − | Y | +++++ | +++++ |
| 6/20/2004 | − | Y | +++++ | +++++ |
| 6/21/2004 | + | N | +++++ | ++++ |
| 6/22/2004 | + | N | ++++ | ++++ |
| 6/23/2004 | + | N | ++++ | +++ |
| 6/24/2004 | + | N | ++++ | +++ |
| 6/25/2004 | + | N | ++++ | +++ |
| 6/26/2004 | − | Y | ++++ | +++++ |
| 6/27/2004 | + | N | ++++ | ++++ |
| 6/28/2004 | + | N | +++ | +++ |
| 6/29/2004 | + | N | +++ | ++ |
| 6/30/2004 | + | N | +++ | ++ |
| 7/1/2004 | + | N | +++ | ++ |
| 7/2/2004 | + | N | +++ | ++ |
| 7/3/2004 | + | N | ++ | + |
| 7/4/2004 | + | N | ++ | + |

*Y: Observation of throwing-outs or vomiting from the dog
N: No observation of throw-outs or vomiting from the dog
**Body odors were determined by the average of three people who objectively smelled the dog twice a day.
+++++: very strong odors; ++++: strong odors; +++: some what strong odors; ++: less strong odors; + some odors.
***Gas released from dogs were observed and the odors were the average of three people; +++++: very strong odors; ++++: strong odors; +++: some what strong odors ++; less strong odors; + some odors

TABLE 1 shows that feeding of a dog daily with probiotics reduced the incidence of vomiting, and reduced odor, in particular, reduced bad body odor, and reduced the incidence of flatulence.

### EXAMPLE 2

Eight Chesapeake Bay Retriever dogs aged from 1 to 13 years with different chronic digestive disorders were treated daily by mixing one MITOMAX capsule with the morning feeding for 28 days. TABLE 2 shows the results. In TABLE 2 the age of the dogs is in years, the weight is in pounds.

TABLE 2

| | | | Initial | Initial | Final | |
|---|---|---|---|---|---|---|
| Dog | Sex | Age | Weight | Symptoms | Weight | Outcome |
| 1 | M | 9 | 85 | lost appetite | 85 | increased |
| | | | | | | appetite, |
| | | | | | | firm stool |
| 2 | F | 6 | 75 | poor digestion | 75 | digestion |
| | | | | | | improved, |
| | | | | | | firm stool |
| 3 | M | 5 | 86 | loose stool, | 86 | firm stool, |
| | | | | diarrhea | | no diarrhea |
| 4 | F | 9 | 80 | poor digestion, | 80 | digestion and |
| | | | | swallowing | | swallowing |
| | | | | difficulty | | improved |
| 5 | M | 13 | 70 | loose stool, | 70 | firm stool, |
| | | | | diarrhea | | no diarrhea |
| 6 | F | 2 | 68 | lost appetite | 68 | improved |
| | | | | | | appetite |
| 7 | F | 1 | 60 | lost appetite, | — | improved |
| | | | | loose stool, | | appetite, |
| | | | | diarrhea | | firm stool, |
| | | | | | | no diarrhea |
| 8 | F | 9 | 80 | vomiting 2 or 3 | 80 | no vomiting |
| | | | | times a week | | |

TABLE 2 shows that daily feeding dogs of both sexes and a variety of ages with a capsule of probiotics resulted in improvement in digestion, in particular in improvement in appetite, reduction of diarrhea and the improvement in firmness of stools, reduction of swallowing difficulty, and reduction of vomiting.

Microencapsulation of lactic acid bacteria using biopolymers.

Viable lactic acid bacteria and yeasts used in probiotics for pets, such as dogs and cats, are encapsulated and protected by the microbial biopolymers xanthan gum and chitosan. Xanthan gum is a polysaccharide gum which dissolves readily in water with stirring to give highly viscous solutions at low concentrations. It forms strong films on evaporation of aqueous solutions and is resistant to heat degradation. Chitin is a polysaccharide consisting predominately of unbranched chains of N-acetyl-glucosamine residues. Chitosan is deacylated chitin, a polymer often used in water treatment, photographic emulsion, in improving the dyeability of synthetic fibers and fabrics and in wound-healing preparations.

Probiotic microbes were encapsulated with an aqueous solution containing 0.5 to 2.5 percent (weight by volume) xanthan gum and 0.2 to 0.8 percent (weight by volume) chitosan. The pH of the solution was from 2.0 to 7.0. A preferred solution contained 1.25 percent (weight by volume) xanthan and 0.4 percent (weight by volume) chitosan at a pH of 4.15. Viable microbial cells are encapsulated at up to 1010 colony forming units (cfu) per ml.

Encapsulation of viable probiotic microbes in the mixture of xanthan gum and chitosan has the advantage of protecting the viability of the microbes, of delivering the proper dosage of viable probiotic microbes to the pet or dog which is being fed, and of facilitating the feeding of the probiotic microbes. Dogs and cats do not reject the probiotic microbes when they are encapsulated in a mixture of xanthan gum and chitosan.

Without wishing to be held to this explanation, the inventors suggest the observed efficacy of the chitosan and xanthan gum solution in encapsulation of probiotic microbes is due to the formation of a xanthan-chitosan complex. The mixture of two oppositely charged polyelectrolytes in aqueous solution results in formation of a polyelectrolyte complex due to the electrostatic attraction of oppositely charged polymers. It is postulated that at moderate pH values the xanthan gum is predominately associated with a large number of net negative charges, while chitosan is associated with a large number of net positive charges. The two polymers with opposite net charges therefore bind together forming a stable complex and a strong gel. Relatively high pH values deionize the amino groups on the chitosan with resulting less stable binding between the two polymers and less strong capsules.

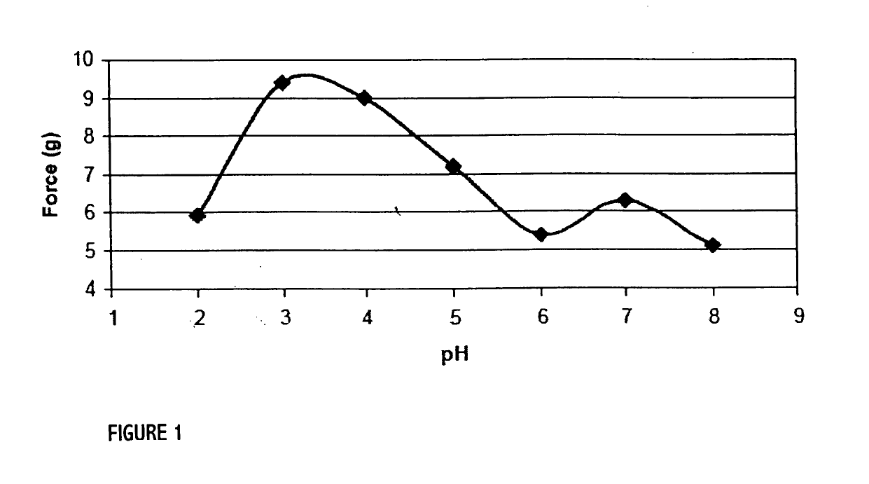

FIG. 1 is a graph showing the capsule hardness at pH values from 2 to 8. Capsules were formed as in the preferred process above. Capsule hardness or mechanical strength was measured at a variety of pH values using TA.XT2i, using a 5 kg load cell and a distance of 1 mm. FIG. 1 showed that the hardness of the capsules peaks in the pH range of 3 to 4, and was relatively low at pH 6 to 8. The data of FIG. 1 are consistent with the above theoretical discussion of the formation of a chitosan-xanthan gum complex.

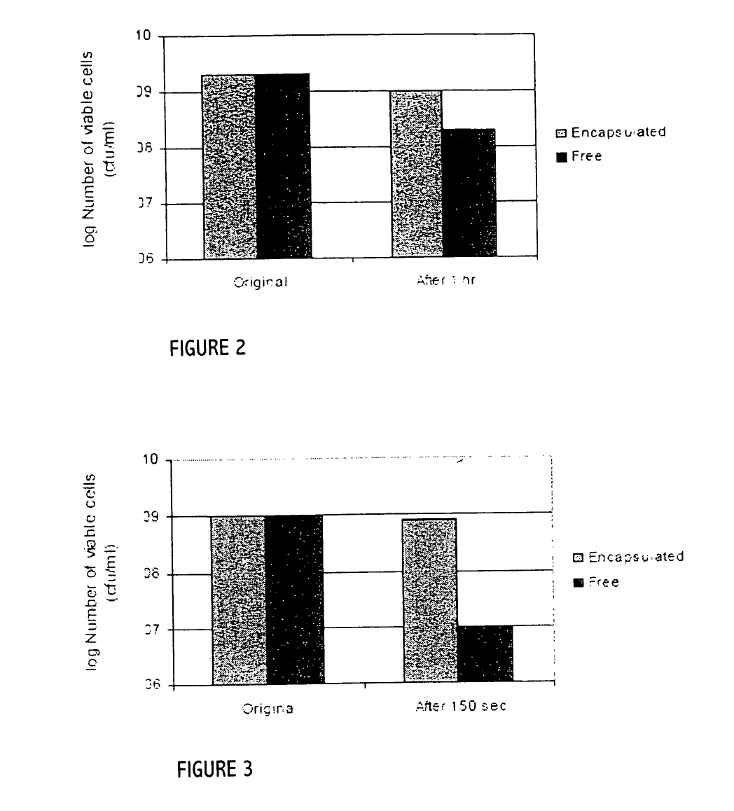

FIG. 2 shows the effect of low temperature on the viability of encapsulated and unencapsulated microbes. Encapsulated and unencapsulated microbes were held for one hour at 0° C. The number of unencapsulated viable microbes declined from about 10^{9.3} cfu/ml to about 10^{8.3} cfu/ml. The number of encapsulated viable microbes declined from about 10^{9.3} cfu/ml to about 10⁹ cfu/ml. FIG. 2 shows the protective effect of encapsulation against low temperature.

FIG. 3 shows the effect of high temperature on the viability of encapsulated and unencapsulated microbes. Encapsulated and unencapsulated microbes were held for 150 seconds at 60° C. The number of unencapsulated viable microbes declined from about 10⁹ cfu/ml to about 10⁷ cfu/ml. The number of encapsulated viable microbes declined from about 10⁹ cfu/ml to about 10^{8.9} cfu/ml. FIG. 3 shows the protective effect of encapsulation against high temperature.

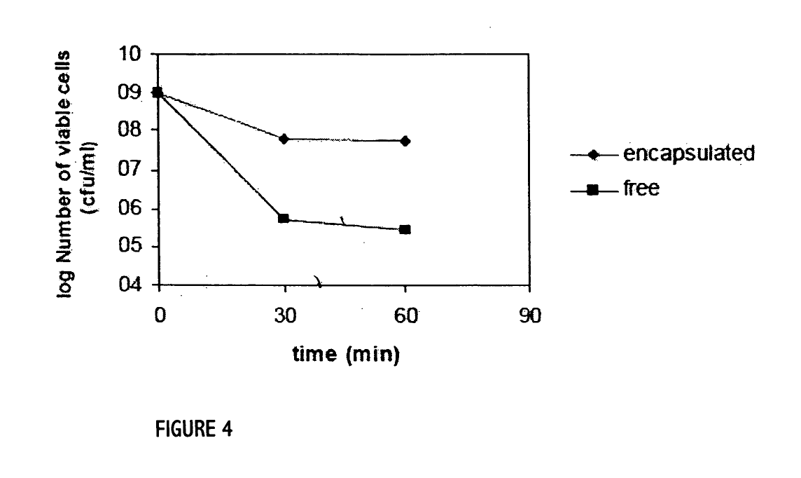

FIG. 4 shows the effect of low pH on the viability of encapsulated and unencapsulated microbes. Encapsulated and unencapsulated microbes were held from 0 to 60 minutes at pH 2. The number of unencapsulated viable microbes declined from about 10⁹ cfu/ml to about 10^{5.7} cfu/ml after 30 minutes and to about 10^{5.5} cfu/ml after 60 minutes. The number of encapsulated viable microbes declined from about 10⁹ cfu/ml to about 10^{7.8} cfu/ml at both 30 and 60 minutes. FIG. 4 shows the protective effect of encapsulation against low pH.

While a number of exemplary aspects and embodiments have been discussed above, those of skill in the art will recognize certain modifications, permutations, additions and sub combinations thereof. It is therefore intended that the following appended claims and claims hereafter introduced are interpreted to include all such modifications, permutations, additions and sub-combinations as are within their true spirit and scope.

We claim:

1. A preparation for pets comprising viable probiotic microbes encapsulated in a mixture of biopolymers.

2. The preparation of claim 1 wherein the probiotic microbes comprise lactic acid bacteria or yeast.

3. The preparation of claim 2 wherein the probiotic microbes comprise Pediococcus, Lactobacillus, Bifidobacterium, Bacillus, Streptococcus or Enterococcus bacteria or Saccharomyces yeast.

4. The preparation of claim 3 wherein the Pediococcus bacteria is Pediococcus acidilactici and the Saccharomyces yeast is Saccharomyces cerevisiae boulardii.

5. The preparation of claim 1 wherein the biopolymers are xanthan and/or chitosan gum.

6. The preparation of claim 5 wherein the xanthan gum concentration is from about 0.2 percent weight by volume to about 2 percent weight by volume and the concentration of chitosan gum is about 0.1 percent weight by volume to 1.0 percent weight by volume and the pH is from about 2 to about 7.

7. The preparation of claim 6 wherein the xanthan gum concentration is from about 01.25 percent weight by volume and the concentration of chitosan gum is about 0.4 percent weight by volume and the pH is about 4.15.

8. The process of improving the digestion of pets in need of such an improvement comprising the step:
feeding the pet viable encapsulated probiotic microbes.

9. The process of claim 8 wherein the probiotic microbes comprise yeast.

10. The process of claim 8 wherein the probiotic microbes comprise lactic acid bacteria.

11. The process of claim 8 wherein the probiotic microbes comprise yeast and lactic acid bacteria.

12. The process of claim 9 wherein the yeast are Saccharomyces yeast.

13. The process of claim 10 wherein the lactic acid bacteria are Pediococcus, Lactobacillus, Bifidobacterium, Bacillus, Streptococcus or Enterococcus bacteria.

14. The process of claim 11 wherein the probiotic microbes comprise Saccharomyces yeast and one or more lactic bacteria from Pediococcus, Lactobacillus, Bifidobacterium, Bacillus, Streptococcus or Enterococcus bacteria.

15. The process of claim 12 wherein the Saccharomyces yeast is Saccharomyces cerevisiae boulardii.

16. The process of claim 13 wherein the Pediococcus bacteria is Pediococcus acidilactici.

17. The process of claim 14 wherein the probiotic microbes are Saccharomyces cerevisiae boulardii and Pediococcus acidilactici.

18. The process of claim 8 wherein the probiotic microbes are encapsulated in a mixture of xanthan gum and chitosan or in gelatin.

19. The process of claim 8 wherein the pet is a dog, a cat, a rabbit, a guinea pig, a hamster, or a bird.

20. The process of claim 8 wherein the improvement in digestion is increased appetite, reduced diarrhea, increased firmness of stool, reduction in vomiting, reduction of body odor, reduction of flatulence, or improved swallowing.

21. The process of selecting lactic acid bacteria with enhanced immune responses comprising the steps:
cultivating bacteria in low pH and high salt media,
feeding chickens the bacteria,
isolating lactic acid bacteria from the chicken feces,
cultivating the isolated lactic acid bacteria with human cell lines, and
isolating lactic acid bacteria which adhere to the human cell lines.

22. The process of claim 21 wherein the lactic acid bacteria are Pediococcus, Bifidobacterium, Bacillus, Streptococcus or Enterococcus bacteria.

23. The process of claim 22 wherein the Pediococcus bacteria is Pediococcus acidilactici.
